(19) United States
(12) Patent Application Publication
(10) Pub. No.: US 2009/0252741 A1
(43) Pub. Date: Oct. 8, 2009

(54) HUMAN MONOCLONAL ANTI-CTLA4 ANTIBODIES IN CANCER TREATMENT

(75) Inventors: Yang Liu, Columbus, OH (US); Pan Zheng, Columbus, OH (US); Kenneth May, JR., Columbus, OH (US); Ken Lute, Winchester, OH (US)

Correspondence Address: CALFEE HALTER & GRISWOLD, LLP, 800 SUPERIOR AVENUE, SUITE 1400, CLEVELAND, OH, 44114, US

(73) Assignee: OHIO STATE UNIVERSITY RESEARCH FOUNDATION, Columbus, OH (US)

(21) Appl. No.: 10/536,113

(22) PCT Filed: Sep. 7, 2005
(86) PCT No.: PCT/US2005/031898

§ 371 Date: Mar. 17, 2008

Related U.S. Application Data
(60) Provisional application No. 60/607,825, filed on Sep. 8, 2004.
(60) Provisional application No. 60/699,464, filed on Jul. 15, 2005.

(51) Int. Cl. A61K 39/395; C07K 16/00; C12Q 1/02; A61P 37/04

(52) U.S. Cl. 424/154.1; 530/388.23; 530/387.3; 435/29

(57) ABSTRACT

Although results from preclinical studies in animal models have proven the concept for use of anti-CTLA-4 antibodies in cancer immunotherapy, two major obstacles have hindered their successful application for human cancer therapy. First, the lack of in vitro correlates of the anti-tumor effect of the antibodies makes it difficult to screen for the most efficacious antibody by in vitro analysis. Second, significant autoimmune side-effects have been observed In a recent clinical trial. In order to address these two issues, we have generated human CTLA-4 gene knock-in mice and used them to compare a panel of anti-human CTLA-4 antibodies for their ability to induce tumor rejection and autoimmunity. Surprisingly, while all antibodies induced protection against cancer and demonstrated some autoimmune side effects, the antibody that induced the strongest protection also induced the least autoimmune side effects. These results demonstrate that autoimmune disease does not quantitatively correlate with cancer immunity. Our approach may be generally applicable to the development of human therapeutic antibodies.

This application claims priority to U.S. Provisional Application No. 60/607,825, filed Sep. 8, 2004, and to U.S. Provisional Application 60/699,464, filed Jul. 15, 2005. The entire disclosure of both of these applications is incorporated herein by reference.

Work leading to this invention was supported, at least in part, by grants from the National Cancer Institute: R01CA69091, R01CA58033, R41CA93107 and P01CA9542-01. The government has certain rights in this invention.

The present invention relates to methods for screening for monoclonal antibodies to CTLA4 that are useful in enhancing T cell response, for example, in cancer treatment. The invention further relates to novel monoclonal antibodies to human CTLA4.

Monoclonal antibodies to CTLA4 can be obtained using conventional techniques. Briefly, antibodies can be obtained by immunizing an animal with at least a portion of the CTLA4 protein. Animals that can be used for this purpose include, but are not limited to, rat, mouse, goat, sheep, hamster, dog, and rabbit. The CTLA4 can be from any mammal, including but not limited to, humans, mice, rats, etc. In some embodiments, the CTLA4 is human CTLA4 and the host animal is mouse. It should be noted that there is considerable sequence identity between many mammalian species and in those instances, cross-species immunoreactivity is anticipated.

The portion of CTLA4 used for creating the monoclonal antibody can be of any fragment, up to the entire protein. In some embodiments, the fragment used is the extracellular domain of CTLA4. In other embodiments, the fragment used is at least 8, 9, 10, 11, 12, 13, 14, 15, 16, 17, 18, 19, 20 or more contiguous amino acids. In certain embodiments, the fragment used is at least 8, 9, 10, 11, 12, 13, 14, 15, 16, 17, 18, 19, 20 or more contiguous amino acids of the extracellular domain of CTLA4. The CTLA4 protein can be expressed alone or as a fusion protein. In some embodiments, the CTLA4 protein is expressed as a fusion protein with the Fc fragment of an immunoglobulin, such as human IgG1.

The spleen and/or lymph nodes of the immunized animal then provide the source of cells for the hybridoma. The isolated cells are fused, for example using polyethylene glycol, with myeloma cells to produce hybridoma cells. The culture supernatant from the hybridomas can then be screened using standard techniques to identify those producing antibodies with the desired specificity.

The hybridoma cells can be grown in culture, or the cells can be injected into animals, such as mouse, rat, etc., for production of monoclonal antibodies to the CTLA4. The antibody may be purified from the hybridoma cell culture supernatants or the injected animals' ascites fluid by conventional techniques.

In some embodiments, it may be desirable to decrease the antigenicity of the monoclonal antibody. This can performed by “humanizing” the antibody.

In some embodiments, it may be desirable to further screen those monoclonal antibodies that will produce a desirable therapeutic effect. This can be achieved by testing the monoclonal antibodies in an SCID mouse model. Briefly, peripheral blood leukocytes (PBL) can be obtained from healthy persons; EBV-seropositive samples are selected. The PBL can be separated from other cell types using conventional techniques, such as a Ficoll gradient. The PBL can then be injected into mice for engraftment. In some embodiments, the SCID mice are CB.17 SCID mice.

The mice can be given injections of a cytokine to deplete the natural killer cells. This depletion can be performed on the day preceding or on the day of engraftment. The SCID mice can then be injected with the monoclonal antibodies to be tested. The antibodies can be in ascites fluid or can be purified. After a sufficient number of repeated antibody injections, the SCID mice spleen cells can be harvested and stimulated with an EBV⁺ cell line or an EBV⁻ cell line. After stimulation, cells can be washed and stained for the different cell types, including for example, CD45, CD8, and CD4.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate one (several) embodiment(s) of the invention and together with the description, serve to explain the principles of the invention.

### BRIEF DESCRIPTION OF THE DRAWINGS

FIG. 1: Anti-human CTLA-4 mAb promotes the engraftment of PBL and expansion of human T cells within 12 days. CB.17 SCID mice were engrafted with 50×10⁶ human PBL, and treated with 100 μg TMβ1 mAb on day 0, 2, and 4, followed by 300 μg anti-human CTLA-4 mAb or Mouse IgG on days 1, 5, and 9, and 3 μg human GM-CSF every other day. At 12 days after engraftment, mice were sacrificed and spleens were harvested for staining. a) Total cellularity within spleens. b) Representative FACS plot showing expanded percentage of CD45⁺, CD4⁺, and CD8⁺ cells. CD4⁺ and CD8⁺ are gated from among CD45⁺ cells. c) Total cell numbers of CD45⁺ (left panel), and CD4⁺ and CD8⁺ cells (right panel). d) Percentage of CD45⁺, CD4⁺, and CD8⁺ cells within live cell gate. All panels are representative of 4-5 mice per treatment group. Bars represent mean plus SEM. P-values were generated using one-way ANOVA with Tukey's procedure for multiple comparisons. Asterisks indicate a difference from the control mouse IgG treatment with a significance of p<0.05.

FIG. 2: Anti-human CTLA-4 mAb promotes the engraftment of PBL and expansion of human T cells at 24 days. CB.17 SCID mice were engrafted with 50×10⁶ human PBL, and treated with 100 μg TMβ1 mAb on days −1, 1, and 3, 100 μL ascites containing anti-human CTLA-4 mAb or 100 μg mouse IgG on days 1, 5, 9, and 13, and 3 μg human GM-CSF every other day. At 24 days after engraftment, mice were sacrificed and spleens were harvested and pooled for staining. a) Representative dot plot showing expansion of CD8 and CD4 T cells with anti-human CTLA-4 mAb clone L3D10 treatment. b) Variable expansion of CD8 and CD4 T cells with treatment by different clones of anti-human CTLA-4 mAb. Bars represent cells from pooled spleens from two to three mice per treatment group.

FIG. 3: Anti-human CTLA-4 mAb L3D10 decreases percentage of engrafted cells expressing EBV latent membrane protein 1 (LMP-1). Mice were treated as described in FIG. 3 legend, except TMβ1 mAb was given on days 0, 2 and 4. Spleens were harvested at day 22 after engraftment and stained for intracellular LMP-1 or isotype IgG2a. a) Representative FACS plot of LMP-1 staining, depicting cells from lymphocyte gate. Data shown are LMP1 and Cd19 profiles of gated human CD45⁺ cells. b) Summary graph showing % (top) and number of LMP-1 staining cells for 3 to 4 individual mice per treatment group. Data are representative of two independent experiments. The p-value was generated using a two-sample t-test.

FIG. 4: Anti-human CTLA-4 mAb L3D10 promotes preferential expansion of lymphoblastoid cell line-reactive CD8 T cells. a) LMP-1 expression by an autologous EBV-positive lymphoblastoid cell line (LCL), and an allogeneic EBV-negative Burkitt's lymphoma cell line, used as stimulators for IFNγ production by hu-PBL-SCID spleen cells. b) CB.17 SCID mice were engrafted with 50×10⁶ human PBL, and treated with 100 μg TMβ1 mAb on the same day, followed by 300 μg anti-human CTLA-4 mAb L3D10 or mouse IgG and 3 μg human GM-CSF on days 1, 5, and 9. Spleen cells were harvested at day 29 after engraftment and stimulated for 6 hours with autologous LCL or allogeneic Burkitt's lymphoma as a control. Samples were then stained for IFNγ-producing CD8 T cells. L3D10-treated mice show an almost 3-fold increase in percentage of IFNγ-producing CD8 T cells with LCL stimulation compared with control mice. Neither treatment group showed reactivity to Burkitt's lymphoma. FACS plots represent pooled spleens from nine mouse IgG-treated and five L3D10-treated mice. Plots shown are within the CD45⁺CD8⁺ gate.

FIG. 5: Some anti-human CTLA-4 mAbs prolong survival and delays onset of lymphoproliferative disorder in hu-PBL-SCID mice. CB.17 SCID mice were engrafted with 50×10⁶ human PBL and treated with 100 μg TMβ1 mAb the same day, followed by 100 μL ascites containing anti-human CTLA-4 mAbs or 100 μg Mouse IgG, and 3 μg human GM-CSF on days 1, 5, and 9, and 13 following engraftment. Mice were monitored for signs of illness and sacrificed when moribund. One L3D10-treated mouse with early death at day 15 and one KM10G11-treated mouse with death on day 13 were excluded from the survival analysis based on our experience that no lymphoma-related death is possible at this point. The survival times of the antibody-treated groups were compared to control Ig-treated group using the log rank test. P values are: Mouse IgG versus L3D10=0.0195; Mouse IgG versus L1B11=0.481; Mouse IgG versus K4G4=0.323; Mouse IgG versus KM10G11=0.045; Mouse IgG versus YL2=0.324.

FIG. 6: Comparison of two anti-CTLA4 antibodies for their effect in tumor rejection. hCTLA4(+/+) mice were challenged with MC38 tumor cells on day. After CTLA4 antibodies (4G6, K4G4, L1B11 or L3D10) or control mouse IgG (mlg) were injected on days 2, 9 and 16 (200 μg/injection/mouse). Tumor sizes were measured every 3 days. Growth of individual tumors in each of the 4 mice per group is depicted in A-E. The means and S.D. of tumor sizes in each group are summarized n in F.

FIG. 7: Anti-human CTLA-4 antibodies with different potency in delaying tumor growth. a) Growth kinetics of MC38 tumors in minimal disease model. CTLA-4(h/h) mice were challenged with MC38 (5×105/mouse) in the lower abdomen. Two days later, the mice received either control mouse IgG or anti-CTLA-4 antibodies K4G4, L1B11 or L3D10 and the tumors were measured every 3-4 days. Data shown represent means and SEM of tumor volumes until day 55 when some mice in antibody treated groups reached their tumor burden endpoint. b) Log transformation of tumor volume. The tumor growth over time was analyzed using Stata'sR XTGEE (cross sectional generalized estimating equations) model. Six tests were done to compare the exponential slopes. All mAbs significantly delayed the growth kinetics of tumors (P<0.001). In addition, significant delay of tumor growth was observed in mice that received L3D10 in comparison to those that received either L1B11 or K4G4 (P<0.001). c) Kaplan-Meier survival curves of mice that received either control IgG or one of the anti-CTLA-4 antibodies. Complete rejection of tumors was observed in 2 out of 9 mice in the L3D10-treated group. A log-rank test revealed that the three mAbs significantly prolonged mouse survival (p=0.0000-0.0038). Data shown in (a) and (b) are representative of those from two independent experiments, involving a total of 8-9 mice per group, those in (c) involve 8-9 mice per group.

FIG. 8: L3D10 treatment delays growth of established tumors in human CTLA-4 knock-in mice. MC38 tumor cells were injected subcutaneously into the human CTLA-4 knock-in mice. At 10-14 days after tumor injection, when the tumors reached a mean diameter of 8 mm, the mice were injected with either L3D10 or control 19 every four days for 4 weeks. a) Growth kinetics of established tumors in mice treated with either control IgG or L3D1 (n=9). Data shown are means and SEM of tumor volumes. The volumes of large holes caused by necrosis in some mice were subtracted. Student t tests were used to compare the tumor size at each time point, those with P<0.05 were indicated with *, while those with P<0.01 were indicated with **. b) Kaplan-Meier survival curves of mice that received control IgG or L3D10. A log-rank test revealed that L3D10 significantly prolonged mouse survival (p=0.011).

FIG. 9: Autoimmune side effects associated with different anti-CTLA-4 antibodies. Serum samples from mice that received anti-CTLA-4 treatment, were collected on days 30 (a) and 55 (b) and tested for anti-dsDNA antibodies. Data shown are means and S.D. of O.D. at 490. c) Correlation between tumor growth suppression and anti-DNA antibodies in control IgG, L1B11 and K4G4, but not in L3D10-treated mice. Data shown are the means and SEM of tumor sizes and O.D.490 of ELISA test using 1:270 dilution of sera from tumor bearing mice. Tumor size and anti-DNA antibody levels reflect data collected at 30 days post tumor challenge. The relative strength of anti-cancer immunity and autoimmunity has been repeated in two independent experiments involving 8-9 mice per group.

FIG. 10. Anti-CTLA-4 antibodies with distinct anti-tumor and autoimmune effects bound to an overlapping site on CTLA-4 and blocked B7-1/CTLA-4 interaction. a-c) Cross-competition. 100 μg/ml of unlabeled anti-CTLA-4 antibodies were added to plates coated with CTLA-4Ig. Given concentration of the biotinylated antibodies were added to the wells after 10 min. The amounts of biotinylated antibodies bound were determined by adsorption of HRP-labeled streptavidin to the plates. Data shown were means and SEM of O.D.490. d) All anti-CTLA-4 antibodies used in the study block B7-1-CTLA-4 interaction. CHO cells transfected with human B7-1 were incubated with a mixture of CTLA-4Ig and given anti-CTLA-4 antibodies. After washing away the unbound antibodies, the binding of CTLA-4Ig was determined by flow cytometry using APC-labeled goat anti-human CTLA-4 antibody. Data shown are histograms depicting CTLA-4Ig binding to human B7-1-transfected CHO cells.

### DESCRIPTION OF THE EMBODIMENTS

Reference will now be made in detail to the present embodiment(s) (exemplary embodiments) of the invention, example(s) of which is (are) illustrated in the accompanying drawings.

### EXAMPLES

### Materials and Methods

Experimental animals BALB/c mice were purchased from Charles River Laboratories under contract with the National Cancer Institute. CB.17 SCID mice and BALB/c RAG-2(−/−) mice were purchased from Taconic (Germantown, N.Y.). All mice were maintained in the University Laboratory Animal Research Facility at the Ohio State University under specific pathogen-free conditions.

Monoclonal antibody production BALB/c mice were immunized two times with a fusion protein consisting of the extracellular domain of the human CTLA-4 protein and the Fc fragment of human IgG1 (huCTLA-4Ig). Spleen cells were harvested from immunized mice and fused with myeloma cell line XAg8.653 using polyethylene glycol (MW 1000) (Sigma, St. Louis, Mo.). Hybridomas were selected in HAT media and further cultured in HT media. Culture supernatant was screened for the presence of anti-human CTLA-4 mAb by ELISA. Clones producing mAb that bound to human CTLA-4Ig fusion protein but not mouse CD28Ig fusion protein were rescreened by ELISA and further subcloned and expanded. Large-scale antibody production of selected clones was achieved by purifying mAb from culture media using a Protein G column or by intraperitoneal injection of 5×10⁶ hybridoma cells into BALB/c RAG-2(−/−) mice to produce ascites. Isotyping of mAb was performed using a kit purchased from BD Pharmingen (San Diego, Calif.).

Binding kinetics and affinity of monoclonal antibodies These experiments were performed by Biacore, Inc. through a contract service. Human CTLA-4Ig fusion protein was immobilized on a Biacore sensor chip using primary amine covalent chemistry. Briefly, N-hydroxysuccinimide esters were introduced on the chip surface by modification of the carboxymethyl groups on the chip surface with a mixture of N-hydroxysuccinimide (NHS) and N-ethyl-N′-(3-dimethylaminopropyl)-carbodiimide hydrochloride (EDC) for 7 minutes. The human CTLA-4Ig was diluted in 10 mM sodium acetate (pH 5.5) at a concentration of 2.5 μg/mL and injected over the activated surface for approximately 1-3 minutes. The surface was then blocked for 7 minutes with ethanolamine to remove any remaining esters. An NHS/EDC activated and ethanolamine blocked surface was used as the reference surface. Anti-human CTLA-4 mAbs were injected at various concentrations in duplicate over the protein and reference surface for 3 minutes, followed by 10 minutes of dissociation time using an automated method. The running buffer was HBS-EP (0.01 HEPES pH 7.4, 0.15 M NaCl, 3 mM EDTA, 0.005% Surfactant P20) pH 7.4 and the detection temperature was 25° C.

Engraftment of human peripheral blood leukocytes PBL were obtained from normal healthy donors that were consented under an IRB-approved protocol for leukapheresis performed by The Ohio State University Hospitals apheresis unit. Selected donors were EBV-seropositive and Hepatitis B and HIV-seronegative. These PBL were previously shown to generate EBV lymphoproliferative disorder in greater than 90% of engrafted hu-PBL-SCID mice. PBL were separated from other cell types using a Ficoll gradient. 50×10⁶ PBL were injected intraperitoneally in 0.5 mL PBS into CB.17 SCID mice.

Monoclonal antibody and cytokine treatment. Mice were given intraperitoneal injections of 100 μg anti-1L2Rβ (TMβ1) mAb to deplete murine NK cells on the day preceding or the day of engraftment. In the experiments analyzing T cell expansion and LMP-1 expression, this initial treatment was followed by two additional treatments of 100 μg of TMβ1 mAb every other day. Mice received intraperitoneal injections of 300 μg purified anti-human CTLA-4 mAb or 100 μl ascites containing anti-human CTLA-4 mAb, or 100-300 μg control mouse IgG (Sigma, St. Louis, Mo.) on days 1, 5, 9, and 13 after PBL engraftment. Mice also received intraperitoneal injections of 3 μg human GM-CSF every other day for 3 weeks. In the experiment assessing IFNγ production, mice received a single dose of 100 μg TMβ1 mAb, followed by 300 μg purified anti-human CTLA-4 mAb or control mouse IgG and 3 μg human GM-CSF on days 1, 5, and 9.

Flow cytometry All antibodies used for staining of cell surface and intracellular proteins, such as CD3, CD4, CD8, CD45, LMP-1, IFNγ, and were purchased from BD Pharmingen (San Diego, Calif.). Intracellular staining for LMP-1 and IFNγ was performed using a Cytofix/CytoPerm kit (BD Pharmingen). Samples were analyzed on a BD FACSCalibur flow cytometer.

IFNγ production assay Hu-PBL-SCID spleen cells were stimulated with an autologous EBV⁺ lymphoblastoid cell line or an allogeneic EBV Burkitt's lymphoma cell line at a 4:1 ratio for 6 hours in the presence of GolgiStop (BD Pharmingen, San Diego, Calif.). After stimulation, cells were washed and stained for extracellular CD45, CD8, and CD4, followed by intracellular staining with IFNγ or isotype IgG1.

Survival experiment CB.17 SCID mice were engrafted with 50×10⁶ PBL and treated with 100 μg of TMβ1 mAb on the same day, followed by 100 μL ascites containing anti-human CTLA-4 mAb or 100 μg mouse IgG and 3 μg human GM-CSF on days 1, 5, 9, and 13. Mice were monitored for signs of illness and sacrificed when moribund. Necropsy was performed to determine the presence of lymphoproliferative disorder or graft-versus-host disease.

Statistical analysis Statistical significance and p-values for T cell expansion experiments were determined using one-way ANOVA with Tukey's procedure for multiple comparisons. The p-value for difference in LMP-1 expression was determined by two-sample t-test. For the survival curve, the mean survival time and standard error of the mean survival time were calculated for each group using the Kaplan-Meier estimate. The survival times of the groups were compared using the log rank test [27].

Results

1. Generation of a panel of mouse anti-human CTLA-4 monoclonal antibodies. BALB/c mice were immunized two times with human CTLA-4Ig fusion protein, consisting of the extracellular domain of human CTLA-4 and the Fc fragment of human IgG1. Spleen cells from these mice were fused with the myeloma cell line XAg8.653. After several fusions, a panel of more than 20 hybridomas producing significant amounts of monoclonal antibody against the human CTLA-4 molecule was generated. Five of these clones were selected for experimentation upon demonstration of significant binding to human CTLA-4 by ELISA. All five of the antibodies were determined to be IgG1,κ isotype, which facilitates direct comparison of any immunologic response that may be mediated by these antibodies. The affinities of each antibody for human CTLA-4Ig fusion protein were measured using a Biacore instrument. As shown in Table 1, the K_{D} of the antibodies ranged from 0.72 nM to 10 nM.

TABLE 1
Binding kinetics and affinity of anti-human CTLA-4 mAb to
huCTLA-4Ig fusion protein (as determined by Biacore).

| Antibody | Avg. kₐ ± SD | Avg. k_{d} ± SD | K_{d} ± SD |
|---|---|---|---|
| clone | (1/Ms) (10⁵) | (1/s) (10⁻⁴) | (nM) (10⁻⁹) |
| L3D10 | 2.07 +/− 0.25 | 6.33 +/− 3.18 | 3.06 +/− 1.70 |
| L1B11 | 1.10 +/− 0.11 | 12.0 +/− 1.32 | 10.9 +/− 1.75 |
| K4G4 | 1.99 +/− 0.27 | 6.82 +/− 2.20 | 3.40 +/− 1.40 |
| KM10 | 2.11 +/− 0.21 | 1.72 +/− 0.34 | 0.82 +/− 0.23 |
| YL2 | 3.61 +/− 0.28 | 2.61 +/− 0.52 | 0.72 +/− 0.20 |

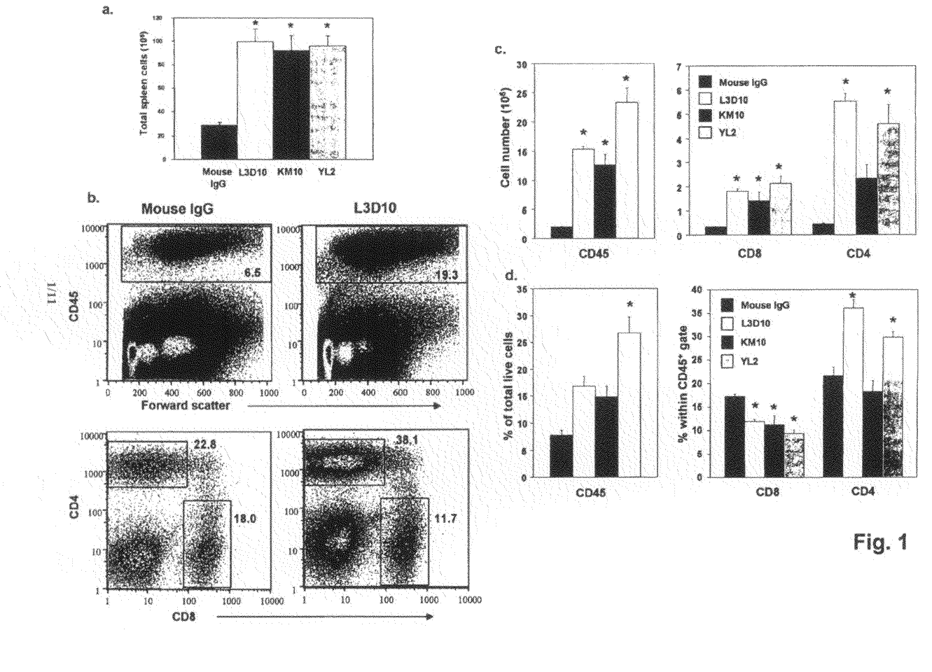

2. Anti-human CTLA-4 mAb promotes a profound expansion of T cells in a hu-PBL-SCID mouse model. To test whether our anti-human CTLA-4 mAb had any biological activity in vivo, the hu-PBL-SCID mouse model was employed. This model provides a unique setting in which the interaction of a functional human immune system with EBV-generated lymphoproliferative disease can be observed [20]. SCID mice were engrafted with human PBL and treated with different clones of anti-human CTLA-4 mAb, plus human GM-CSF to promote the generation and maturation of antigen-presenting cells [28]. As shown in FIG. 1a, at 12 days after injection of human PBL, all three anti-human CTLA-4 antibodies increased the total number of splenocytes by more than 3-fold compared with control mice (p<0.002). In addition, a selective expansion of human leukocytes, as marked by expression of human CD45, was observed among all antibody-treated mice (p<0.003) (FIG. 1b top panel, c & d left panels). The total number of CD8 T cells was increased in all antibody-treated groups (p<0.03), while total number of CD4 T cells was increased in L3D10 and YL2-treated groups (p<0.0003) (FIG. 1b, lower panel and FIG. 1c). However, at this time point, the antibodies differ in their ability to selectively expand human T cell subsets. First, in mice that received L3D10 (p<0.0003) and YL2 (p<0.02), the proportion of CD4 T cells expanded significantly in comparison to those treated with control IgG. In contrast, KM10 did not cause any preferential expansion (p=0.52) (FIG. 1b lower panels and 1d). The proportion of CD8 T cells among human leukocytes decreased significantly (p<0.009) even as the total numbers increased (FIGS. 1c and d). A comparison between FIGS. 1a and 1c revealed that the numbers of mouse cells were also significantly increased, perhaps in response to cytokines induced by anti-CTLA4 antibodies.

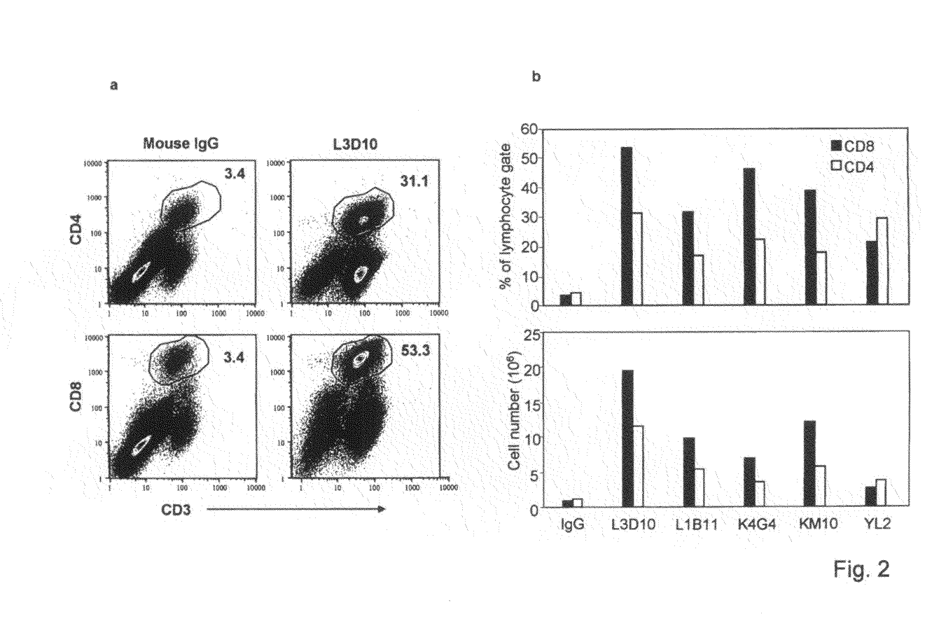

At the third week after reconstitution, all five anti-CTLA-4 antibodies were analyzed for their effect on the number of human CD4 and CD8 T cells in the spleen. An example is given in FIG. 2a and the comparison of the different antibodies is presented in FIG. 2b. As shown in FIG. 2a, L3D10 caused a more than a 10-fold expansion of CD4 and CD8 T cells. Interestingly, the five clones of anti-human CTLA-4 mAb displayed differential effects not only on the amount of T cell expansion, but also on the relative effect on CD4 versus CD8 T cell subsets. Most clones of anti-human CTLA-4 mAb showed a preferential expansion of CD8 T cells at this time point, while one clone of mAb showed a slightly preferential increase in CD4 T cells. These data clearly demonstrate that the inventive anti-human CTLA-4 mAbs promote the expansion of human T cells and increase the engraftment of total human PBL in the hu-PBL-SCID mouse model.

Surprisingly, the extent of T cell expansion did not correlate with the binding affinities of the antibodies to human CTLA-4 (Table 1). The two antibodies with highest affinities, KM10 and YL2, did not induce the greatest T cell expansion, and even varied from one another in their ability to induce T cell expansion despite very similar K_{D} values. Since anti-human CTLA-4 mAb clone L3D10 showed the greatest effect in expanding human T cells, this clone was chosen for further characterization.

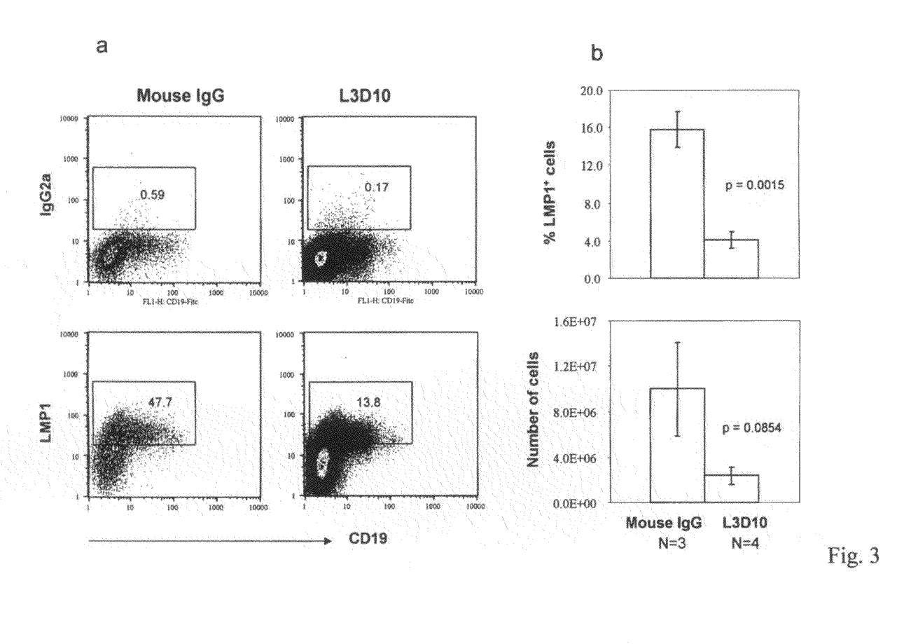

3. Anti-human CTLA-4 mAb decreases EBV-mediated transformation of human cells. When PBL from EBV-seropositive donors are used in the hu-PBL-SCID mouse model, transformation of PBL by EBV promotes the development of lymphoproliferative disease. Latent membrane protein 1 (LMP-1) is an EBV oncoprotein involved in the immortalization of B cells leading to this transformation [29-33], and LMP-1 has been shown to be a potential target of T cell responses [34, 35]. Hence, the number of cells expressing LMP-1 can be taken as a reflection of the number of cells that have been transformed by EBV and could undergo oncogenesis. One way to determine whether the expansion of T cells mediated by anti-human CTLA-4 mAb treatment has any therapeutic effect is to examine the level of LMP-1 being expressed within engrafted cells. To test this, SCID mice were engrafted with PBL and treated with anti-human CTLA-4 mAb L3D10 or mouse IgG. Mice were sacrificed 22 days after engraftment, and spleen cells were analyzed. Similar to the experiment shown in FIG. 2, a substantial expansion of both CD8 and CD4 T cell subsets was observed (data not shown). As shown in FIGS. 3a and b, the percentage of LMP-1⁺ cells was 34 fold lower in mice treated with L3D10 (p=0.0015). Similar magnitude of reduction was observed in the total number of LMP1⁺ cells in the spleens. However, large intra-group variation reduced the p value to 0.0854. These results suggest that the percentage of EBV-infected cells can be reduced as a result of anti-CTLA-4 mAb treatment. Interestingly, while essentially all CD19⁺ cells expressed LMP1, the majority of the LMP1⁺ cells lacked CD19 marker. The origin of these LMP1⁺CD19⁺ cells is unclear at this stage.

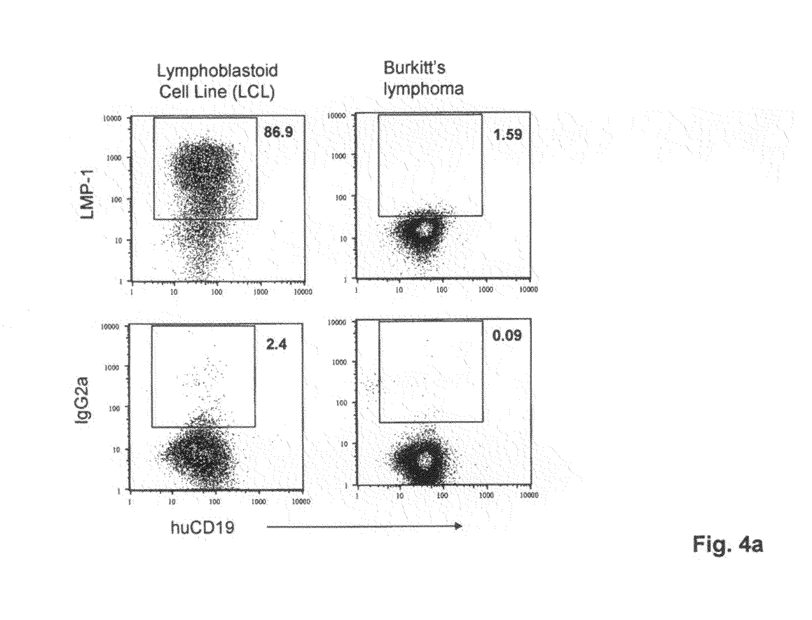

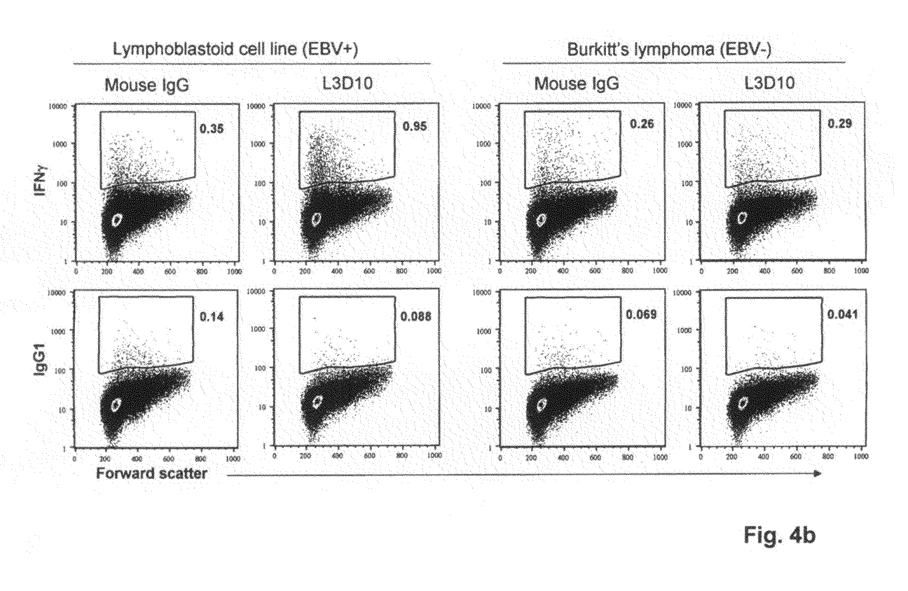

4. Anti-human CTLA-4 mAb promotes expansion of LCL-reactive CD8 T cells. To test whether antigen-specific T cells were induced in our model, we stimulated spleen cells harvested from hu-PBL-SCID mice with an autologous EBV-positive lymphoblastoid cell line (LCL) or allogeneic EBV-negative Burkitt's lymphoma for 6 hours in vitro, and evaluated IFNγ production by CD8 T cells. The LCL was generated from a tumor harvested from a hu-PBL-SCID mouse previously engrafted with the same donor's PBL. To verify the expression level of EBV protein, these stimulator cell types were stained for intracellular LMP-1 expression. As shown in FIG. 4a, almost all the LCL cells expressed high levels of intracellular LMP-1, while the Burkitt's lymphoma cells had minimal or no LMP-1 expression. After 6 hours of stimulation with these cells, nearly a 3-fold increase in the percentage of IFNγ-producing CD8 T cells was observed in L3D10-treated mice compared with control mice (FIG. 4b). This indicates that anti-human CTLA-4 mAb can promote the preferential expansion of antigen-specific CD8 T cell responses, as well as promoting overall expansion of T cells.

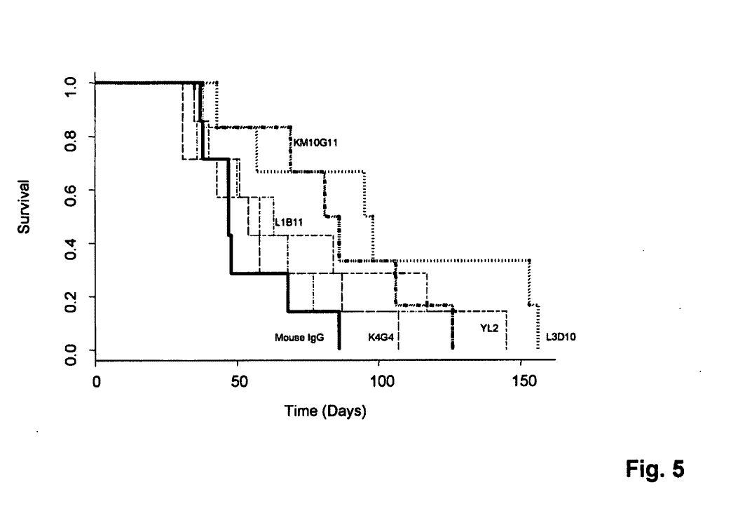

5. Anti-human CTLA-4 mAb L3D10 delays the development of lymphoproliferative disease in hu-PBL-SCID mice. A long-term survival experiment was performed in which engrafted mice were treated with anti-human CTLA-4 mAbs and human GM-CSF for two weeks after engraftment, and then observed for signs of illness. FIG. 5 shows the survival curve of mice that received control Ig or one of five different anti-CTLA-4 antibodies. Based on our previous experience that no lymphoproliferative diseases can be observed within one months of engraftment, we have excluded small number of mice that died before 30 days from final analysis. L3D10 mediated an almost two-fold extension in the mean survival time of engrafted mice compared with control mice (100.3+/−17.5 days with L3D10 versus 53.0+/−6.2 days with mouse IgG), which was statistically significant (p=0.0195). In addition, KM10G11 also had an statistically significant impact (p=0.045). However, while it appears that other anti-CTLA4 antibodies also extended the life span of recipient mice somewhat, pair-wise comparisons demonstrate no statistical difference between these antibodies and control IgG.

Discussion

Here is described the use of a hu-PBL-SCID mouse model to obtain a more thorough preclinical screening of anti-human CTLA-4 mAb to identify the most efficacious clones from a panel of mAbs. The hu-PBL-SCID mouse model was first described by Mosier et al. as a method to reconstitute a functional human immune system in SCID mice by intraperitoneal injection of human peripheral blood leukocytes [20]. This report described the long-term engraftment of all cellular components of the human immune system, and also observed the spontaneous development of human B cell lymphomas when PBL from Epstein Barr virus (EBV)-seropositive donors were used. These lymphomas were subsequently characterized as being similar to the large cell lymphomas observed in immunosuppressed transplant patients [37], also known as post-transplant lymphoproliferative disorder (PTLD). Since the initial reports, numerous groups have utilized this model to test various aspects of immune function and lymphomagenesis, and in the process, discovered a number of limitations of this model, including xenograft-versus-host disease (XGVHD), variations in PBL engraftment, and leakiness of the SCID phenotype [38-42]. Despite these caveats, the hu-PBL-SCID model remains one of the few mouse models with which to assess spontaneous human tumor development and the resultant anti-tumor immune response. More recently, evidence has accumulated that the control of EBV-lymphoproliferative disorder is mediated by CD8 cytotoxic T lymphocytes both in patients with PTLD [43, 44] and hu-PBL-SCID mice [26, 45]. With the identification of EBV latent and lytic antigens, it has been demonstrated that specific CD8 T cell responses to these EBV antigens can be detected in seropositive human patients [34, 46, 47] and in hu-PBL-SCID mice [26]. Correlation of CD8 T cell responses to protection against EBV-lymphoproliferative disorder in hu-PBL-SCID mice makes this model valuable for the study of anti-human CTLA-4 mAb.

This Example clearly demonstrates the ability of anti-human CTLA-4 mAb to mediate dramatic expansion of CD8 and CD4 T cell populations. In conjunction, mAb promotes the overall engraftment or survival of human PBL in the SCID mouse. Interestingly, each clone of anti-human CTLA-4 mAb possessed varying ability to promote T cell expansion and PBL engraftment. Since all antibodies were of the same isotype, these variations cannot be attributed to isotype difference. In addition, the affinity of the antibodies used does not adequately explain the functional heterogeneity. For instance, two pairs of antibodies with essentially identical affinity showed different function in vivo. This lack of in vitro and in vivo correlation warrants the use of more stringent preclinical screening regimens such as the one described here to select clones of mAb that can elicit the most dramatic in vivo T cell response.

It has been reported that human T cells engrafted in SCID mice represent an anergic phenotype and that once anergy is broken, most reactivity of CD4 T cells is directed against mouse antigens [48]. It is possible that the lack of T cell expansion in our control mice was due to an anergic state of the T cells, and that treatment with anti-human CTLA-4 mAb was sufficient to reverse this anergy and permit T cell expansion. This has important implications for a tumor setting in which T cells might be tolerized to tumor antigen, as been demonstrated in at least one mouse model [11].

Not only did anti-human CTLA-4 mAb promote overall T cell expansion in vivo, but several different parameters suggested that the robust T cell expansion with anti-human CTLA-4 mAb treatment had therapeutic value. The first was a significant decrease in the percentage of cells that expressed intracellular LMP-1. As an EBV oncoprotein that is critical for the generation of lymphoma, LMP-1 may be viewed as a surrogate marker for the potential formation of tumor within the mice. Reduced levels at an early time point before lymphoproliferative disease normally appears, reveals the impact of mAb treatment in reducing the oncogenic source of tumor.

Secondly, a preferential expansion of antigen-specific CD8 T cells with anti-human CTLA-4 mAb treatment was observed. This enhanced expansion was elicited in mice treated with a more limited GM-CSF regimen than that used in experiments showing overall T cell expansion and LMP-1 reduction. Interestingly, the mice treated with L3D10 under the limited GM-CSF regimen did not show overall expansion of T cells compared with control mice, despite their preferential increase in antigen-specific CD8 T cells. Additional experiments using frozen spleen cells from mice treated with L3D10 and the more extensive GM-CSF protocol (every other day) showed enhanced overall T cell expansion but not antigen-specific expansion when compared with control mice. It is difficult to directly compare the use of fresh and frozen cells for in vitro stimulation due to the decreased viability and increased background staining associated with thawed cells, but perhaps the interaction of anti-CTLA-4 mAb and GM-CSF is more complicated than predicted in the hu-PBL-SCID mouse model.

Thirdly, in a longer-term experiment a prolongation of survival was observed with anti-human CTLA-4 mAb treatment, providing another piece of evidence that mAb can promote anti-tumor immune responses. This result must be taken with caution, as this experiment and other attempts to reproduce the finding were complicated by the development of severe illness, most likely XGVHD, which sometimes caused death before lymphoma formation in a substantial fraction of mice involved. However, in mice that escaped XGVHD, the trend of prolonged survival is intriguing. Taken together these data show an important role for anti-human CTLA-4 mAb in the expansion of human T cells and the promotion of immunity against a spontaneous virally induced tumor. Furthermore, variability in the efficacy of different clones of mAb warrants the use of novel models such as this one, to provide more thorough preclinical screening of candidate mAb for clinical translation.

Screening Antibodies In CTLA4 Knock-In Mice

As an alternative approach to compare the potency of anti-human CTLA-4 antibodies, mice that are homozygous for human CTLA-4 gene knock-in are used. Such mice are described in U.S. application Ser. No. 09/957,688, published as 2002/0115209.

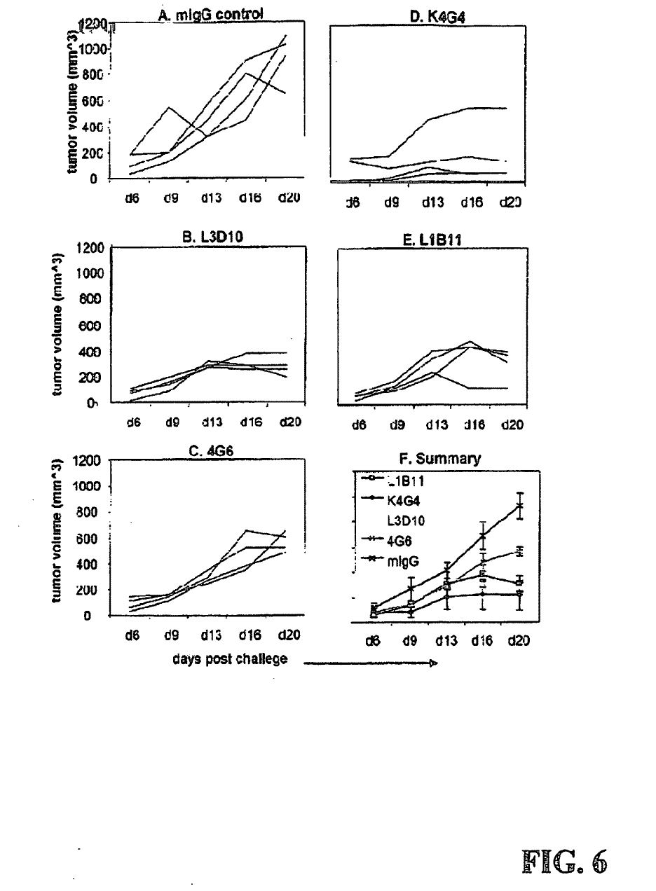

As Shown in FIG. 6, in human CTLA4 knock-in mice, 3 of 4 anti-CTLA4 antibodies tested significantly reduced the growth rate of tumors. The most potent antibodies in this case is K4G4, which halted growth of tumor growth in 3 of the 4 mice tested.

Tumorigenicity Assay. MC38 cells (5×105) suspended in serum free RPMI (100 μl) were injected s.c. in the lower abdomen of mice. For the minimal disease model mice were treated once a week beginning on day two. In the established disease model, mice were treated every four days with treatments beginning 10-14 days post-challenge. In both models the tumor-bearing mice received identical doses of either anti-human CTLA-4 mAb or control mouse IgG (200 □g/mouse/injection). Tumor size and incidence were determined every 2-5 days by physical examination. The tumor volume was calculated using an established formula of volume=½(long×short2). All mice were sacrificed when the tumor volume reached 4000 mm³. The number of days required for tumors to reach this endpoint was used for survival analysis.

Detection of anti-double stranded DNA antibodies. Anti-DNA antibodies were measured by ELISA.

Immunofluorescence for antibody and complement deposition in the kidney glomerulus Frozen sections of kidney were prepared from euthanized mice and fixed in acetone. After blocking with 10% normal goat serum, the sections were stained with Rhodamine-conjugated goat anti-mouse IgG and FITC-conjugated goat anti-mouse C3 antibodies (ICN Biomedicals, Inc.)

Results

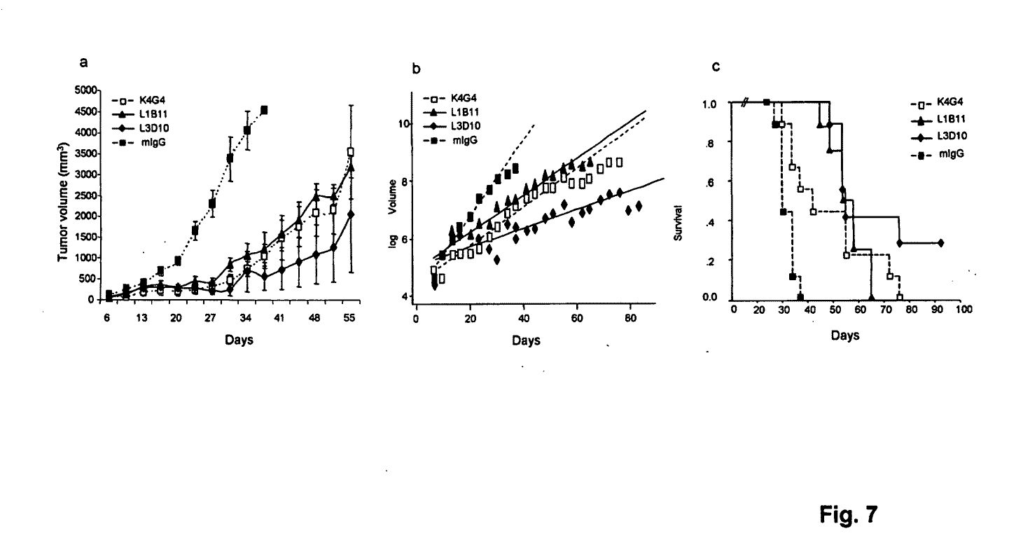

1. The human CTLA-4 knock-in mice discriminate therapeutic effects of anti-CTLA-4 antibodies with essentially Identical affinity and isotype. To test whether human CTLA-4 knock-in mice are useful in discriminating the therapeutic effect of the anti-CTLA-4 antibodies, colon cancer cell line MC38 was injected subcutaneously into the CTLA-4 knock-in mice. Two days later, the tumor cell-bearing mice received either control IgG or one of three isotype-matched anti-CTLA-4 antibodies. Among them, L3D10 and K4G4 have the same affinity and binding kinetics, while L1B11 has approximately 3-fold lower affinity. As shown in FIG. 7a, all three antibodies demonstrated a statistically significant delay in tumor growth compared with mouse IgG control antibody. In addition, L3D10 proved to be the most potent antibody when compared to the other two treatment antibodies (FIGS. 7a, b). As seen in FIG. 7c all three antibodies led to enhanced survival compared to control Ig-treated mice. A survival advantage of L3D10-treated mice was also observed over those treated with LB11 and K4G4 (FIG. 7c).

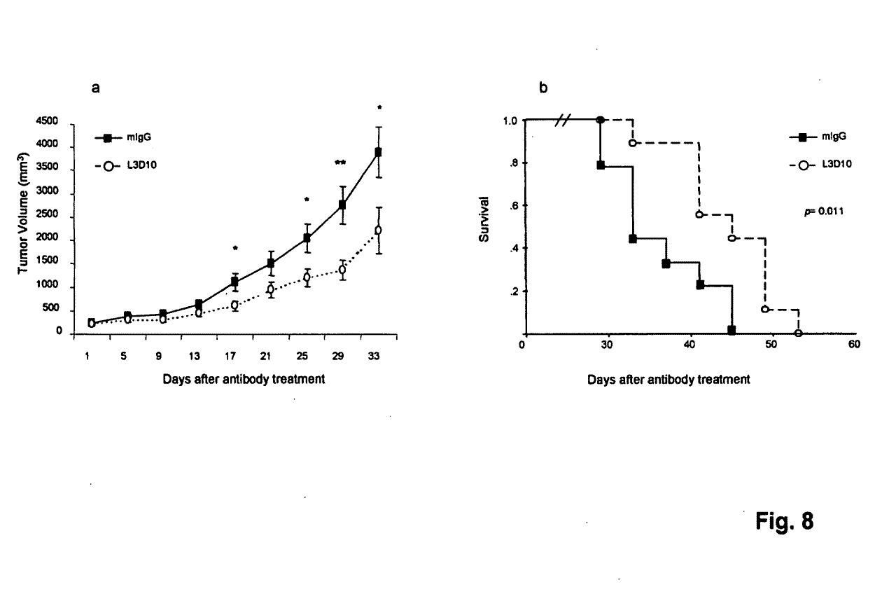

To explore the therapeutic potential of the L3D10 antibody for large established tumors, we delayed treatment until approximately 2 weeks after tumor cell challenge. As shown in FIG. 8, in comparison to the control Ig-treated group, the L3D10 antibody delayed tumor growth, and prolonged survival of tumor-bearing mice.

2. The human CTLA-4 knock-in mice unravel the link between cancer immunity and autoimmunity. Given the tendency of anti-CTLA-4 antibodies to exacerbate autoimmune diseases in experimental autoimmune models, it is of interest to determine whether the autoimmune side effects quantitatively correlate with anti-tumor immunity. Our analysis revealed that in wild-type mice, anti-mouse CTLA-4 antibody 4F10 suppressed tumor growth, but enhanced anti-double stranded DNA antibodies. In contrast, anti-4-1BB antibody 2A induced cancer immunity without triggering anti-DNA antibody response. Thus, the anti-DNA antibodies can serve as a useful marker for autoimmunity associated with anti-CTLA-4 antibody.

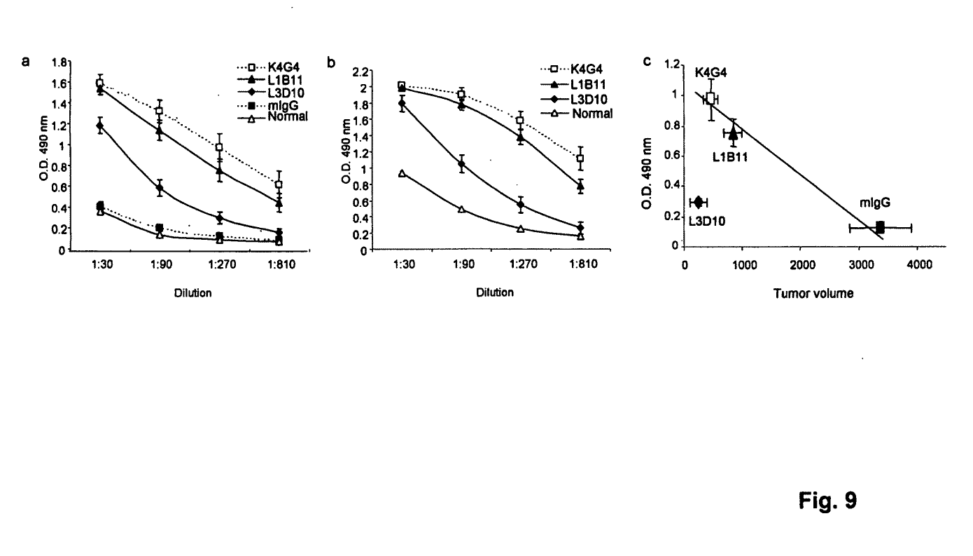

Mice treated with three different anti-CTLA-4 antibodies were compared for their production of anti-double stranded (ds) DNA antibodies. As shown in FIG. 9a and FIG. 9b, although anti-dsDNA antibodies were detected in all tumor bearing mice treated with anti-CTLA-4 antibodies, the mice that received K4G4 and L1B11 had 3-5-fold higher levels of anti-dsDNA antibodies than mice treated with L3D10. The difference was stable over the course of the treatment. Consistent with this variability in anti-dsDNA antibody induction, more IgG deposition in kidney glomeruli of K4G4 or L1B11-treated mice was observed compared with mice treated with L3D10:

Antibody and complement C3 deposition in kidney glomeruli are shown in Table 2, below.

TABLE 2

| | K4G4 | L1B11 | L3D10 | mIgG |
|---|---|---|---|---|
| IgG | 4/8* | 4/8* | 2/9 | 0/9 |
| C3 | 0/8 | 0/8 | 1/9 | 0/9 |

Frozen section of kidney were analyzed after the mice were euthanized when they reach early removal criteria (tumors reach 4000 mm³), with exception of 2 mice in the L3D10-treated group in which tumors never reached the criteria for early removal. The incidences of IgG deposition in mice treated K4G4 (P=0.029) and L1B11 (P=0.029), but not L3D10 (P=0.47), are significantly higher than the control group.

A comparison between the amounts of the anti-dsDNA antibody and the sizes of the tumors suggests that for mice that received control Ig, L1B11 or K4G4, tumor size correlated inversely with the amounts of anti-dsDNA antibodies. This observation suggests that, among these 3 groups, the intensity of the anti-tumor immune response correlates with that of the anti-DNA antibody response (FIG. 9c). However, the group that received L3D10-treatment had the smallest tumor size with the lowest anti-dsDNA antibody levels. Thus, stronger cancer immunity does not have to be coupled with more severe autoimmune side-effects.

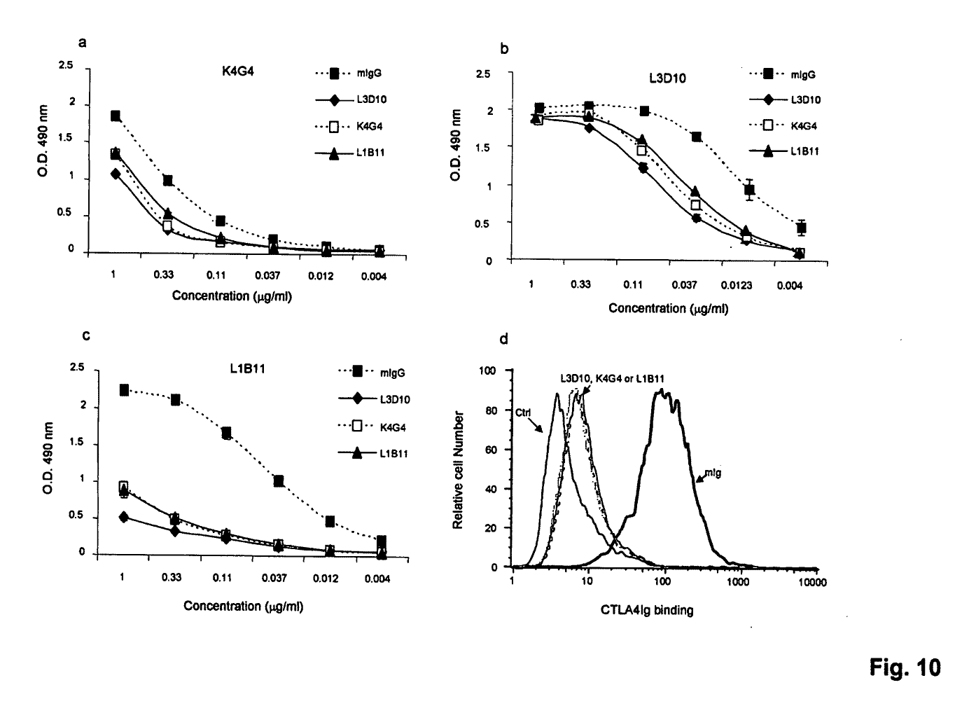

3. Anti-CTLA-4 antibodies that induce different potencies in anti-tumor and autoimmune response bind to an overlapping site on CTLA-4. As measured by Biacore, L3D10 and K4G4 have essentially identical affinity for human CTLA-4 23. In addition, these antibodies have identical isotype (IgG1, κ). To determine whether the antibodies have overlapping binding sites, we tested whether they compete with each other in binding to human CTLA-4. As shown in FIGS. 10a-c, all three antibodies cross-blocked each other's binding to CTLA-4, with efficiency that grossly correlates with their affinity to CTLA-4. Moreover, all antibodies were capable of blocking the binding of CTLA-4 to its natural ligand B7-1 (FIG. 10d). The similarity of the immunochemical properties of these antibodies highlights the need for preclinical models to screen for anti-CTLA4 antibodies with favorable therapeutic activity and acceptable autoimmune side effect.

Discussion

The human CTLA-4 gene knock-in mice can serve as a valuable model for the preclinical screening of cancer therapeutic antibodies targeting the human CTLA-4 protein. A model that recapitulates autoimmune side-effects will not only allow us to select antibodies with fewer side effects, but also develop approaches to abrogate remaining side effects. The discordance between cancer immunity and autoimmunity reveals that autoimmune side-effects and cancer therapeutic effects are not quantitatively linked. Such uncoupling provides a theoretical basis for selecting optimal anti-CTLA-4 antibodies or other therapeutic agents with the most desirable balance between cancer immunity and autoimmunity.

Other embodiments of the invention will be apparent to those skilled in the art from consideration of the specification and practice of the invention disclosed herein. It is intended that the specification and examples be considered as exemplary only, with a true scope and spirit of the invention being indicated by the claims.
1. Leach, D. R., M. F. Krummel, and J. P. Allison, Enhancement of antitumor immunity by CTLA-4 blockade. Science, 1996. 271(5256): p. 1734-6.
2. Kwon, E. D., et al., Manipulation of T cell costimulatory and inhibitory signals for immunotherapy of prostate cancer. Proc Natl Acad Sci U S A, 1997. 94(15): p. 8099-103.
3. Kwon, E. D., et al., Elimination of residual metastatic prostate cancer after surgery and adjunctive cytotoxic T lymphocyte-associated antigen 4 (CTLA-4) blockade immunotherapy. Proc Natl Acad Sci USA, 1999. 96(26): p. 15074-9.
4. Hurwitz, A. A., et al., Combination immunotherapy of primary prostate cancer in a transgenic mouse model using CTLA-4 blockade. Cancer Res, 2000. 60(9): p. 2444-8.
5. van Elsas, A., A. A. Hurwitz, and J. P. Allison, Combination immunotherapy of B16 melanoma using anti-cytotoxic T lymphocyte-associated antigen 4 (CTLA-4) and granulocyte/macrophage colony-stimulating factor (GM-CSF)-producing vaccines induces rejection of subcutaneous and metastatic tumors accompanied by autoimmune depigmentation. J Exp Med, 1999. 190(3): p. 355-66.
6. van Elsas, A., et al., Elucidating the autoimmune and antitumor effector mechanisms of a treatment based on cytotoxic T lymphocyte antigen 4 blockade in combination with a B16 melanoma vaccine: comparison of prophylaxis and therapy. J Exp Med, 2001. 194(4): p. 481-9.
7. Sutmuller, R. P., et al., Synergism of cytotoxic T lymphocyte-associated antigen 4 blockade and depletion of CD25(+) regulatory T cells in antitumor therapy reveals alternative pathways for suppression of autoreactive cytotoxic T lymphocyte responses. J Exp Med. 2001. 194(6): p. 823-32.
8. Yang, Y. F., et al., Enhanced induction of antitumor T-cell responses by cytotoxic T lymphocyte-associated molecule-4 blockade: the effect is manifested only at the restricted tumor-bearing stages. Cancer Res, 1997. 57(18): p. 4036-41.
9. Hurwitz, A. A., et al., CTLA-4 blockade synergizes with tumor-derived granulocyte-macrophage colony-stimulating factor for treatment of an experimental mammary carcinoma. Proc Natl Acad Sci USA, 1998. 95(17): p. 10067-71.
10. Sotomayor, E. M., et al., In vivo blockade of CTLA-4 enhances the priming of responsive T cells but fails to prevent the induction of tumor antigen-specific tolerance. Proc Natl Acad Sci USA, 1999. 96(20): p. 11476-81.
11. Shrikant, P., A. Khoruts, and M. F. Mescher, CTLA-4 blockade reverses CD8+ T cell tolerance to tumor by a CD4⁺ T cell- and IL-2-dependent mechanism. Immunity, 1999. 11(4): p. 483-93.
12. Hodi, F. S., et al., Biologic activity of cytotoxic T lymphocyte-associated antigen 4 antibody blockade in previously vaccinated metastatic melanoma and ovarian carcinoma patients. Proc Natl Acad Sci USA, 2003. 100(8): p. 4712-7.
13. Phan, G. Q., et al., Cancer regression and autoimmunity induced by cytotoxic T lymphocyte-associated antigen 4 blockade in patients with metastatic melanoma. Proc Natl Acad Sci USA, 2003. 100(14): p. 8372-7.
14. Walunas, T. L., et al., CTLA-4 can function as a negative regulator of T cell activation. Immunity, 1994. 1(5): p. 405-13.
15. Krummel, M. F. and J. P. Allison, CD28 and CTLA-4 have opposing effects on the response of T cells to stimulation. J Exp Med, 1995. 182(2): p. 459-65.
16. Anderson, D. E., et al., Paradoxical inhibition of T-cell function in response to CTLA-4 blockade; heterogeneity within the human T-cell population. Nat Med, 2000. 6(2): p. 2114.
17. McCune, J. M., et al., The SCID-hu mouse: murine model for the analysis of human hematolymphoid differentiation and function. Science, 1988. 241(4873): p. 1632-9.
18. Kamel-Reid, S. and J. E. Dick, Engraftment of immune-deficient mice with human hematopoietic stem cells. Science, 1988. 242(4886): p. 1706-9.
19. Su, L., et al., HIV-1-induced thymocyte depletion is associated with indirect cytopathogenicity and infection of progenitor cells in vivo. Immunity, 1995. 2(1): p. 25-36.
20. Mosier, D. E., et al., Transfer of a functional human immune system to mice with severe combined immunodeficiency. Nature, 1988. 335(6187): p. 256-9.
21. Feuerer, M., et al., Therapy of human tumors in NOD/SCID mice with patient-derived reactivated memory T cells from bone marrow. Nat Med, 2001. 7(4): p. 452-8.
22. Sabzevari, H. and R. A. Reisfeld, Human cytotoxic T-cells suppress the growth of spontaneous melanoma metastases in SCID/hu mice. Cancer Res, 1993. 53(20): p. 4933-7.
23. Stenholm, A. C., A. F. Kirkin, and J. Zeuthen, In vivo eradication of an established human melanoma by an in vitro generated autologous cytotoxic T cell clone: a SCID mouse model. Int J Cancer, 1998. 77(3): p. 476-80.
24. Carballido, J. M., et al., Generation of primary antigen-specific human T- and B-cell responses in immunocompetent SCID-hu mice. Nat Med, 2000. 6(1): p. 103-6.
25. Cochlovius, B., et al., Recombinant gp100 protein presented by dendritic cells elicits a T-helper-cell response in vitro and in vivo. Int J Cancer, 1999. 83(4): p. 547-54.
26. Baiocchi, R. A., et al., GM-CSF and IL-2 induce specific cellular immunity and provide protection against Epstein-Barr virus lymphoproliferative disorder. J Clin Invest, 2001. 108(6): p. 887-94.
27. Klein, J. P., Moeschberger, M. L., Survival Analysis: Techniques for Censored and Truncated Data. 1997, New York: Springer.
28. Markowicz, S. and E. G. Engleman, Granulocyte-macrophage colony-stimulating factor promotes differentiation and survival of human peripheral blood dendritic cells in vitro. J Clin Invest, 1990. 85(3): p. 955-61.
29. Wang, D., D. Liebowitz, and E. Kieff, An EBV membrane protein expressed in immortalized lymphocytes transforms established rodent cells. Cell, 1985. 43(3 Pt 2): p. 831-40.
30. Henderson, S., et al., Induction of bcl-2 expression by Epstein-Barr virus latent membrane protein 1 protects infected B cells from programmed cell death. Cell, 1991. 65(7): p. 1107-15.
31. Fries, K. L., W. E. Miller, and N. Raab-Traub, Epstein-Barr virus latent membrane protein 1 blocks p53-mediated apoptosis through the induction of the A20 gene. J Virol, 1996. 70(12): p. 8653-9.
32. Kulwichit, W., et al., Expression of the Epstein-Barr virus latent membrane protein 1 induces B cell lymphoma in transgenic mice. Proc Natl Acad Sci USA, 1998. 95(20): p. 11963-8.
33. Uchida, J., et al., Mimicry of CD40 signals by Epstein-Barr virus LMP1 in B lymphocyte responses. Science, 1999. 286(5438): p. 300-3.
34. Khanna, R., et al., Identification of cytotoxic T cell epitopes within Epstein-Barr virus (EBV) oncogene latent membrane protein 1 (LMP1): evidence for HLA A2 supertype-restricted immune recognition of EBV-infected cells by LMP1-specific cytotoxic T lymphocytes. Eur J Immunol, 1998. 28(2): p. 451-8.
35. Meij, P., et al., Identification and prevalence of CD8(+) T-cell responses directed against Epstein-Barr virus-encoded latent membrane protein 1 and latent membrane protein 2. Int J Cancer, 2002. 99(1): p. 93-9.
36. Keler, T., et al., Activity and safety of CTLA-4 blockade combined with vaccines in cynomolgus macaques. J Immunol, 2003. 171(11): p. 6251-9.
37. Rowe, M., et al., Epstein-Barr virus (EBV)-associated lymphoproliferative disease in the SCID mouse model: implications for the pathogenesis of EBV-positive lymphomas in man. J Exp Med, 1991. 173(1): p. 147-58.
38. Carlsson, R., et al., Human peripheral blood lymphocytes transplanted into SCID mice constitute an in vivo culture system exhibiting several parameters found in a normal humoral immune response and are a source of immunocytes for the production of human monoclonal antibodies. J Immunol, 1992. 148(4): p. 1065-71.
39. Williams, S. S., et al., Engraftment of human peripheral blood leukocytes into severe combined immunodeficient mice results in the long term and dynamic production of human xenoreactive antibodies. J Immunol, 1992. 149(8): p. 2830-6.
40. Murphy, W. J., D. D. Taub, and D. L. Longo, The huPBL-SCID mouse as a means to examine human immune function in vivo. Semin Immunol, 1996. 8(4): p. 233-41.
41. Amadori, A., et al., The hu-PBL-SCID mouse in human lymphocyte function and lymphomagenesis studies: achievements and caveats. Semin Immunol, 1996. 8(4): p. 249-54.
42. Bankert, R. B., N. K. Egilmez, and S. D. Hess, Human-SCID mouse chimeric models for the evaluation of anti-cancer therapies. Trends Immunol, 2001. 22(7): p. 386-93.
43. Khatri, V. P., et al., Endogenous CD8+ T cell expansion during regression of monoclonal EBV-associated posttransplant lymphoproliferative disorder. J Immunol, 1999. 163(1): p. 500-6.
44. Porcu, P., et al., Successful treatment of posttransplantation lymphoproliferative disorder (PTLD) following renal allografting is associated with sustained CD8(+) T-cell restoration. Blood, 2002. 100(7): p. 2341-8.
45. Rencher, S. D., et al., Activity of transplanted CD8+ versus CD4⁺ cytotoxic T cells against Epstein-Barr virus-immortalized B cell tumors in SCID mice. Transplantation, 1994. 58(5): p. 629-33.
46. Murray, R. J., et al., Identification of target antigens for the human cytotoxic T cell response to Epstein-Barr virus (EBV): implications for the immune control of EBV-positive malignancies. J Exp Med, 1992. 176(1): p. 157-68.
47. Tan, L. C., et al., A re-evaluation of the frequency of CD8+ T cells specific for EBV in healthy virus carriers. J Immunol, 1999. 162(3): p. 1827-35.
48. Tary-Lehmann, M., et al., Anti-SCID mouse reactivity shapes the human CD4+ T cell repertoire in hu-PBL-SCID chimeras. J Exp Med, 1994. 180(5): p. 1817-27.

What is claimed is:

1. A method of identifying monoclonal antibodies to CTLA4 that exhibit an ability to promote enhanced T cell responses, comprising:
administering to an SCID mouse an effective concentration of a monoclonal antibody to CTLA4; and
measuring the population of at least one T cell type chosen from CD4 T cells and CD8 T cells.

2. The method according to claim 1, wherein the SCID mice are engrafted with human peripheral blood leukocytes.

3. The method according to claim 1, wherein the monoclonal antibodies to CTLA4 are derived from clones chosen from L3D10, L1B11, K4G4, KM10, and YL2.

4. A method of enhancing a T cell response in a patient in need thereof, comprising administering an effective amount of at least one CTLA4 monoclonal antibody derived from a clone chosen from L3D10, L1B11, K4G4, KM10, and YL2.

5. The method according to claim 4, wherein the CTLA4 monoclonal antibody is derived from clone L3D10.

6. The method according to claim 4, wherein the antibody is effective to agonize or antagonize CTLA4 signaling.

7. The method according to claim 4, wherein the patient in need thereof is a patient chosen from one diagnosed with cancer, one diagnosed with chronic viral infections, and one anticipating or having undergone organ transplant.

8. A composition for enhancing a T cell response in a patient comprising at least one CTLA4 monoclonal antibody derived from a clone chosen from L3D10, L1B11, K4G4, KM10, and YL2, and at least one pharmaceutically acceptable excipient.

9. The composition according to claim 8, wherein the at least one CTLA4 monoclonal antibody is derived from clone L3D10.

10. The composition according to claim 8, wherein the composition is formulated as an immunization adjuvant.

11. The composition according to claim 8, wherein the at least one CTLA4 monoclonal antibody is derived from clone K4G4.

12. The composition according to claim 8, wherein the at least one CTLA4 monoclonal antibody is derived from clone L3D10 and exhibits anti-dsDNA antibody production below the threshold required to trigger autoimmune disease.

13. The method according to claim 5, wherein the monoclonal antibody is administered to enhance the response of CD8 T cells.

14. A method for enhancing a T cell response in a patient in need of the same comprising administering at least one CTLA4 monoclonal antibody derived from a clone chosen from L3D10, L1B11, K4G4, KM10, and YL2.

15. The method according to claim 14, comprising administering at least one CTLA4 monoclonal antibody derived from clone L3D10 that exhibits anti-dsDNA antibody production below the threshold required to trigger autoimmune disease.

16. The method according to claim 14, comprising administering the anized form of at least one CTLA4 monoclonal antibody derived from clone that exhibits anti-dsDNA antibody production below the threshold required autoimmune disease.
